(19) United States
(12) Patent Application Publication
(10) Pub. No.: US 2006/0106111 A1
(43) Pub. Date: May 18, 2006

(54) Phenylnaphthalene compounds

(76) Inventors: Sophie Poissonnier-Durieux, Rainneville (FR); Said Yous, Loos (FR); Daniel Lesieur, Gondecourt (FR); Caroline Bennejean, Charenton-Le-Pont (FR); Philippe Delagrange, Issy-Les-Moulineaux (FR); Pierre Renard, Le Chesnay (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 10/534,116

(22) PCT Filed: Nov. 4, 2003
(86) PCT No.: PCT/FR03/03278

§ 371 Date: May 5, 2005

(30) Foreign Application Priority Data
Nov. 7, 2002 (FR) 02/13917

(51) Int. Cl. A61K 31/17; A61K 31/165

(52) U.S. Cl. 514/596; 514/617; 564/123; 564/48; 514/621

(57) ABSTRACT

The invention relates to compounds of formula (I): 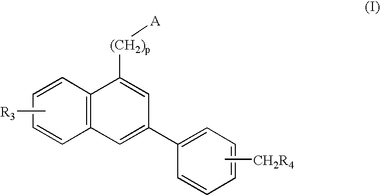 wherein:
A represents 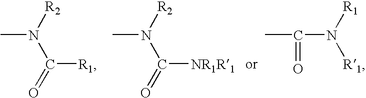
R₃ represents alkoxy,
R₄ is as defined in the description, p is 1, 2 or 3, and medicinal products containing the same which are useful in treating or preventing melatoninergic disorders.

The present invention relates to new phenylnaphthalene compounds, to a process for their preparation and to pharmaceutical compositions containing them.

The compounds of the present invention are new and have pharmacological characteristics that are of great interest in relation to melatoninergic receptors.

In the last ten years, numerous studies have demonstrated the major role played by melatonin (N-acetyl-5-methoxytryptamine) in a large number of physiopathological phenomena and in the control of the circadian rhythm, but melatonin has a rather short half-life owing to the fact that it is rapidly metabolised. Great interest therefore lies in the possibility of making available to the clinician melatonin analogues that are metabolically more stable and have an agonist or antagonist character and of which the therapeutic effect may be expected to be superior to that of the hormone itself.

In addition to their beneficial action in respect of circadian rhythm disorders (J. Neurosurg. 1985, 63, pp. 321-341) and sleep disorders (Psychopharmacology, 1990, 100, pp. 222-226), ligands of the melatoninergic system have valuable pharmacological properties in respect of the central nervous system, especially anxiolytic and antipsychotic properties (Neuropharmacology of Pineal Secretions, 1990, 8 (3-4), pp. 264-272), and analgesic properties (Pharmacopsychiat., 1987, 20, pp. 222-223), and also for the treatment of Parkinson's disease (J. Neurosurg. 1985, 63, pp. 321-341) and Alzheimer's disease (Brain Research, 1990, 528, pp. 170-174). The compounds have also demonstrated activity in relation to certain cancers (Melatonin—Clinical Perspectives, Oxford University Press, 1988, pp. 164-165), ovulation (Science 1987, 227, pp. 714-720), diabetes (Clinical Endocrinology, 1986, 24, pp. 359-364), and in the treatment of obesity (International Journal of Eating Disorders, 1996, 20 (4), pp. 443-446).

Those various effects are exerted via the intermediary of specific melatonin receptors. Molecular biology studies have demonstrated the existence of a number of receptor sub-types that are capable of binding that hormone (Trends Pharmacol. Sci., 1995, 16, p. 50 WO 97.04094). It has been possible for some of those receptors to be located and characterised for different species, including mammals. In order to be able to understand the physiological functions of those receptors better, it is of great advantage to have available selective ligands. Moreover such compounds, by interacting selectively with one or another of those receptors, may be excellent medicaments for the clinician in the treatment of pathologies associated with the melatoninergic system, some of which have been mentioned above.

In addition to being new, the compounds of the present invention exhibit a very strong affinity for melatonin receptors and/or a selectivity for one or another of the melatoninergic binding sites.

The present invention relates, more especially, to the compounds of formula (I): 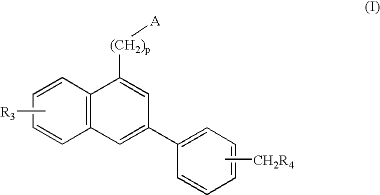
wherein:

A represents a grouping

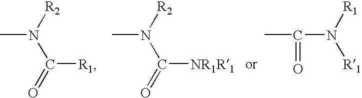

(wherein R₁ and R′₁, which may be identical or different, each represents a linear or branched (C₁-C₆)alkyl group, a linear or branched (C₂-C₆)alkenyl group, a linear or branched (C₂-C₆)alkynyl group, a (C₃-C₈)cycloalkyl group, a (C₃-C₈)cycloalkyl-(C₁-C₆)alkyl group in which the alkyl moiety may be linear or branched, an aryl group, an aryl-(C₁-C₆)alkyl group in which the alkyl moiety may be linear or branched, a heteroaryl group or a heteroaryl-(C₁-C₆)alkyl group in which the alkyl moiety may be linear or branched,

and R₂ represents a hydrogen atom or a linear or branched (C₁-C₆)alkyl group, it being possible, additionally, for R₁ and R₂ together to form a linear or branched alkylene chain containing from 3 to 6 carbon atoms),

R₃ represents a linear or branched (C₁-C₆)alkoxy group,

R₄ represents a halogen atom, a hydroxy group, a linear or branched (C₁-C₆)alkoxy group or an amino group optionally substituted by one or two linear or branched (C₁-C₆)alkyl groups,

p is 1, 2 or 3,

it being understood that:

“aryl” denotes a phenyl, naphthyl or biphenyl group,

“heteroaryl” denotes any mono- or bi-cyclic aromatic group containing from 1 to 3 hetero atoms selected from oxygen, sulphur and nitrogen,

wherein the aryl and heteroaryl groups so defined may be substituted by from 1 to 3 groups selected from linear or branched (C₁-C₆)alkyl, linear or branched (C₁-C₆)alkoxy, hydroxy, carboxy, formyl, nitro, cyano, linear or branched polyhalo(C₁-C₆)alkyl, alkoxycarbonyl and halogen atoms,

to their enantiomers and diastereoisomers, and to addition salts thereof with a pharmaceutically acceptable acid or base.

Among the pharmaceutically acceptable acids there may be mentioned, by way of non-limiting example, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned by way of non-limiting example sodium hydroxide, potassium hydroxide, triethylamine, tert-butylamine etc.

Preferred compounds of the invention are compounds of formula (I) wherein A represents a grouping

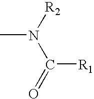

Advantageously, R₁ represents a linear or branched (C₁-C₆)alkyl group such as, for example, a methyl, ethyl, n-propyl or n-butyl group, or a linear or branched (C₃-C₈)cycloalkyl group, such as, for example, a cyclopropyl or cyclobutyl group.

R₂ preferably represents a hydrogen atom.

The preferred value of p is 2.

The preferred R₃ group is the methoxy group.

R₄ advantageously represents an OH, methoxy or NH₂ group, or a halogen atom, such as bromine or iodine.

The preferred position on the phenyl nucleus for the group —CH₂R₄ is the 3 position (or meta).

Even more especially, the invention relates to the following compounds of formula (I): N-(2-{3-[3-(hydroxymethyl)phenyl]-7-methoxy-1-naphthyl} ethyl)acetamide and N-(2-{3-[3-(aminomethyl)phenyl]-7-methoxy-1-naphthyl} ethyl)acetamide.

The enantiomers, diastereoisomers and also addition salts with a pharmaceutically acceptable acid or base of the preferred compounds of the invention form an integral part of the invention.

The present invention relates also to a process for the preparation of the compounds of formula (I), which process is characterised in that there is used as starting material a compound of formula (II): 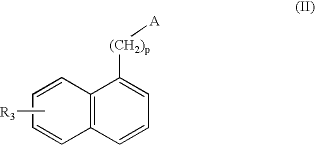
wherein A, p and R₃ are as defined for formula (I), which is subjected to the action of bromine to yield a compound of formula (III): 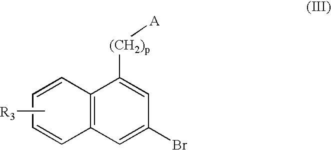
wherein A, p and R₃ are as defined hereinabove, which is condensed, in the presence of palladium acetate or tetrakis(triphenylphosphine)palladium, with a compound of formula (IV): 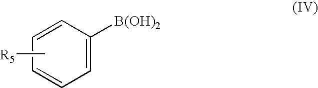
wherein R₅ represents a linear or branched (C₁-C₆)alkoxycarbonyl group, a formyl group or a cyano group, to yield a compound of formula (V): 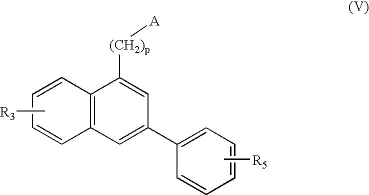
wherein A, p, R₃ and R₅ are as defined hereinabove, which compound of formula (V),

when R₅ represents a CN group, is subjected to the action of Raney nickel to obtain a compound of formula (I/a), a particular case of the compounds of formula (1): 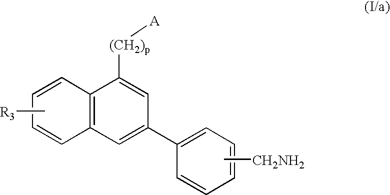
wherein A, p and R₃ are as defined hereinabove,
which compound of formula (I/a) may be subjected to the action of one or more alkylating agents to yield a compound of formula (I/b), a particular case of the compounds of formula (I): 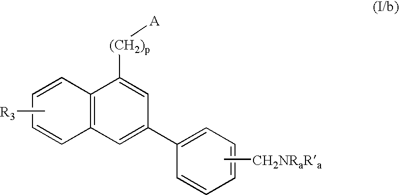
wherein A, p and R₃ are as defined hereinabove, Rₐ represents an alkyl group and R′ₐ represents a hydrogen atom or an alkyl group,

when R₅ represents a formyl group, is subjected to the action of NaBH₄ or triethylsilane and, when R₅ represents an alkoxycarbonyl group, is subjected to the action of LiAlH₄, to yield a compound of formula (I/c), a particular case of the compounds of formula (I): 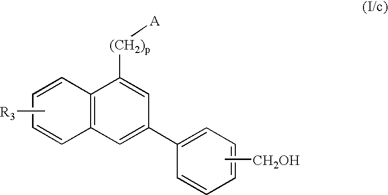
wherein A, p and R₃ are as defined hereinabove, which compound of formula (I/c) is subjected to the action of a hydrohalic acid to yield a compound of formula (I/d), a particular case of the compounds of formula (I): 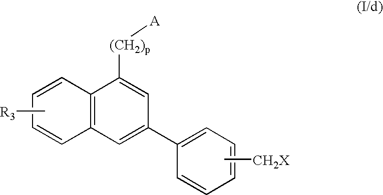
wherein A, p and R₃ are as defined hereinabove and X represents a halogen atom,
or which compound of formula (I/c) is subjected to the action of an alcoholate to yield a compound of formula (I/e), a particular case of the compounds of formula (I): 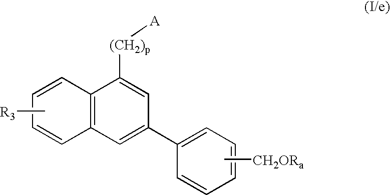
wherein A, p, R₃ and Rₐ are as defined hereinabove,
the compounds (I/a) to (I/e) constituting the totality of the compounds of formula (I), which compounds may be purified according to a conventional separation technique, are converted, if desired, into addition salts with a pharmaceutically acceptable acid or base, and are optionally separated into their isomers according to a conventional separation technique.

The compounds of formula (II) are either commercially available or are obtainable by the person skilled in the art by conventional chemical reactions described in the literature.

In particular, obtaining compounds of formula (II) is described, for example, in the patent specifications EP 0 447 285 and EP 0 745 584.

The invention relates also to compounds of formula (V′): 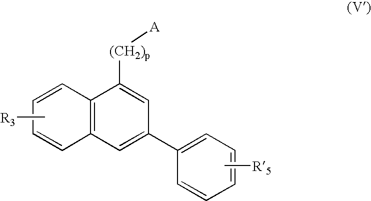
wherein A, p and R₃ are as defined for formula (I) and R′₅ represents a linear or branched (C₁-C₆)alkoxycarbonyl group or a formyl group,
to their enantiomers and diastereoisomers, and to addition salts thereof with a pharmaceutically acceptable acid or base, for use as synthesis intermediates for the preparation of compounds of formula (I) but also as melatoninergic receptor ligands.

A pharmacological study of the compounds of the invention has in fact demonstrated that they are non-toxic, have a very high selective affinity for melatonin receptors and have substantial activity in respect of the central nervous system and, in particular, they have been found to have therapeutic properties in respect of sleep disorders, anxiolytic, antipsychotic and analgesic properties and properties in respect of microcirculation, enabling it to be established that the compounds of the invention are useful in the treatment of stress, sleep disorders, anxiety, seasonal affective disorders or severe depression, cardiovascular pathologies, pathologies of the digestive system, insomnia and fatigue due to jetlag, schizophrenia, panic attacks, melancholia, appetite disorders, obesity, insomnia, psychotic disorders, epilepsy, diabetes, Parkinson's disease, senile dementia, various disorders associated with normal or pathological ageing, migraine, memory losses, Alzheimer's disease, and in cerebral circulation disorders. In another field of activity, it appears that the compounds of the invention can be used in the treatment of sexual dysfunctions, that they have ovulation-inhibitor and immunomodulator properties and that they are capable of being used in the treatment of cancers.

The compounds will preferably be used in the treatment of seasonal affective disorders, severe depression, sleep disorders, cardiovascular pathologies, pathologies of the digestive system, insomnia and fatigue due to jetlag, appetite disorders and obesity.

For example, the compounds will be used in the treatment of seasonal affective disorders, severe depression and sleep disorders.

The present invention relates also to pharmaceutical compositions comprising at least one compound of formula (I) or one compound of formula (V′) on its own or in combination with one or more pharmaceutically acceptable excipients.

Amongst the pharmaceutical compositions according to the invention there may be mentioned more especially those which are suitable for oral, parenteral, nasal, per- or trans-cutaneous, rectal, perlingual, ocular or respiratory administration, especially tablets or dragées, sublingual tablets, sachets, paquets, gelatin capsules, glossettes, lozenges, suppositories, creams, ointments, dermal gels and drinkable or injectable ampoules.

The dosage varies according to the sex, age and weight of the patient, the route of administration, the nature of the therapeutic indication or any associated treatments, and ranges from 0.01 mg to 1 g per 24 hours in one or more administrations.

The following Examples illustrate the invention but do not limit it in any way. The following Preparations result in synthesis intermediates for use in the preparation of the compounds of the invention.

### Preparation 1: N-[2-(3-Bromo-7-methoxy-1-naphthyl)ethyl]acetamide

N-[2-(7-methoxy-1-naphthyl)ethyl]acetamide (29 mmol) is dissolved in 160 ml of acetic acid. The mixture is heated to 70° C. and bromine (35 mmol) is added dropwise in solution in 20 ml of acetic acid. After stirring for 6 hours at that temperature, the reaction mixture is cooled and then poured into iced water. After stirring vigorously for 30 minutes, the mixture is extracted with ethyl acetate. The ethyl acetate phase is dried over magnesium sulphate and then evaporated under reduced pressure. The residue obtained is recrystallised from toluene to yield the title product in the form of a beige solid.

Melting point: 103-105° C.

### Preparation 2: N-[2-(3-Bromo-7-methoxy-1-naphthylethyl]pronanamide

The procedure is as in Preparation 1, with the replacement of N-[2-(7-methoxy-1-naphthyl)ethyl]acetamide with N-[2-(7-methoxy-1-naphthyl)ethyl]propanamide. The title product is recrystallised from 95° ethanol and isolated in the form of a white solid.

Melting point: 146-148° C.

### Preparation 3: N-[2-(3-Bromo-7-methoxy-1-naphthyl)ethyl]butanamide

The procedure is as in Preparation 1, with the replacement of N-[2-(7-methoxy-1-naphthyl)ethyl]acetamide with N-[2-(7-methoxy-1-naphthyl)ethyl]butanamide. The title product is recrystallised from 95° ethanol and isolated in the form of a white solid.

Melting point: 86-88° C.

### Preparation 4: N-[2-(3-Bromo-7-methoxy-1-naphthyl)ethyl]cyclobutanecarboxamide

The procedure is as in Preparation 1, with the replacement of N-[2-(7-methoxy-1-naphthyl)ethyl]acetamide with N-[2-(7-methoxy-1-naphthyl)ethyl]cyclobutanecarboxamide. The title product is recrystallised from 95° ethanol and isolated in the form of a white solid.

Melting point: 154-155° C.

### Preparation 5: 1-[2-(3-Bromo-7-methoxy-1-naphthyl)ethyl]-2-pyrrolidinone

The procedure is as in Preparation 1, with the replacement of N-[2-(7-methoxy-1-naphthyl)ethyl]acetamide with N-[2-(7-methoxy-1-naphthyl)ethyl]-2-pyrrolidinone. The title product is recrystallised from 95° ethanol and isolated in the form of a white solid.

Melting point: 137-139° C.

### EXAMPLE 1

### N-(2-{3-[2-(Hydroxymethyl)phenyl]-7-methoxy-1-naphthyl}ethyl)-acetamide

### Step A: N-{2-[3-(2-Formylphenyl)-7-methoxy-1-naphthyl]ethyl}acetamide

The compound obtained in Preparation 1 (6.2 mmol) is dissolved in 30 ml of toluene and the solution is placed under a stream of nitrogen for 10 minutes. Tetrakis-(triphenylphosphine)palladium (0.25 mmol) is added to the solution and the mixture is again left under a stream of nitrogen for 10 minutes. Sodium carbonate (27 mmol), dissolved beforehand in 10 ml of water, and 2-formylphenylboronic acid (6.8 mmol), dissolved beforehand in 6 ml of ethanol, are added to the mixture. The reaction mixture is heated at reflux for 12 hours and then cooled to ambient temperature, filtered and taken up in 50 ml of water and 50 ml of ethyl acetate. The two phases are separated and the organic phase is dried over magnesium sulphate and evaporated under reduced pressure. The residue obtained is purified by flash chromatography on silica gel (acetone/cyclohexane 2/8) to yield the title product in the form of a pale yellow oil.

### Step B: N-(2-{3-[2-(Hydroxymethyl)phenyl]-7-methoxy-1-naphthyl}ethyl)acetamide

The compound obtained in Step A (2.9 mmol) is dissolved in 40 ml of methanol. Sodium borohydride (5.8 mmol) is then added in small portions and the solution is stirred at ambient temperature for 10 minutes. The methanol is evaporated off and the residue obtained is taken up in an aqueous 1N hydrochloric acid solution and then extracted with ethyl acetate. The organic phase is dried over magnesium sulphate and then evaporated under reduced pressure. The residue is recrystallised from cyclohexane to yield the title product in the form of a pale yellow solid.

Melting point: 57-59° C.

### EXAMPLE 2

### N-(2-{3-[3-(Hydroxymethyl)phenyl]-7-methoxy-1-naphthyl}ethyl)-acetamide

### Step A: N-{2-[3-(3-Formylphenyl)-7-methoxy-1-naphthyl]ethyl}acetamide

The procedure is as in Step A of Example 1, with the replacement of 2-formylphenyl-boronic acid with 3-formylphenylboronic acid. The title product is obtained, after purification by chromatography on silica gel (acetone/cyclohexane: 3/7), in the form of a white solid which is recrystallised from 95° ethanol.

Melting point: 123-125° C.

Elemental microanalysis:

| | % C | % H | % N |
|---|---|---|---|
| calculated | 76.06 | 6.09 | 4.03 |
| found | 75.76 | 6.10 | 4.01 |

### Step B: N-(2-{3-[3-(Hydroxymethyl)phenyl]-7-methoxy-1-naphthyl}ethyl)acetamide

The procedure is as in Step B of Example 1 starting from the compound obtained in

Step A. After purification by chromatography on silica gel (acetone/cyclohexane: 3/7), the title product is obtained in the form of a white solid, which is recrystallised from 95° ethanol.

Melting point: 153-155° C.

Elemental microanalysis:

| | % C | % H | % N |
|---|---|---|---|
| calculated | 75.62 | 6.63 | 4.01 |
| found | 75.33 | 6.61 | 4.22 |

### EXAMPLE 3

### N-(2-{3-[4-(Hydroxymethyl)phenyl]-7-methoxy-1-naphthyl}ethyl)-acetamide

### Step A: Methyl 4-{4-[2-(acetylamino)ethyl]-6-methoxy-2-naphthyl}benzoate

The compound obtained in Preparation 1 (25 mmol), 4-(methoxycarbonyl)phenylboronic acid (27 mmol), palladium acetate (0.05 mmol), sodium hydrogen carbonate (49 mmol) and tetrabutylammonium bromide (0.3 mmol) are dissolved in a dioxane/water mixture (60 ml/40 ml). The mixture is heated at reflux for 4 hours, and then cooled to ambient temperature. 150 ml of ethyl acetate are added and the two phases are separated. The organic phase is dried over magnesium sulphate and evaporated under reduced pressure. The residue obtained is purified by chromatography on silica gel (acetone/cyclohexane: 3/7) to yield the title product in the form of a white solid, which is recrystallised from 95° ethanol.

Melting point: 147-149° C.

### Step B: N-(2-{3-[4-(Hydroxymethyl)phenyl]-7-methoxy-1-naphthyl}ethyl)acetamide

The compound obtained in Step A (5.5 mmol) is dissolved in 30 ml of ether and 10 ml of THF. The solution is cooled to 0° C., and then lithium aluminium hydride (16.5 mmol) is added in small portions. The mixture is stirred at ambient temperature for 6 hours and then the lithium aluminium hydride is hydrolysed by a few drops of aqueous 20% sodium hydroxide solution until a white precipitate is obtained. After filtration, the ether and the THF are evaporated off under reduced pressure and the residue is purified by chromatography on silica gel (acetone/cyclohexane: 3/7) to yield the title product in the form of white solid, which is recrystallised from 95° ethanol.

Melting point: 164-166° C.

### EXAMPLE 4

### N-(2-{3-[3-(Bromomethyl)phenyl]-7-methoxy-1-naphthyl}ethyl)acetamide

The compound obtained in Example 2 (0.6 g; 1.7 mmol) is dissolved in 10 ml of glacial acetic acid and 3.1 ml (17 mmol) of a 45% hydrobromic acid solution in acetic acid. The mixture is stirred at ambient temperature for 24 hours and then poured into 30 ml of iced water. The precipitate formed is filtered off, suctioned off and then recrystallised from 95° ethanol to yield the title product in the form of a pale yellow solid.

Melting point: 118-120° C.

Elemental microanalysis:

| | % C | % H | % N |
|---|---|---|---|
| calculated | 64.09 | 5.38 | 3.40 |
| found | 63.92 | 5.37 | 3.42 |

### EXAMPLE 5

### N-(2-{3-[3-(Iodomethyl)phenyl]-7-methoxy-1-naphthyl}ethyl)acetamide

The compound obtained in Example 4 (0.35 g; 0.85 mmol) is dissolved in 20 ml of acetone and then 0.14 g (0.94 mmol) of sodium iodide are added to the solution. The mixture is heated at reflux with vigorous stirring for two hours. After cooling, the reaction mixture is filtered and then the acetone is evaporated off under reduced pressure. The residue is taken up in water and then extracted with ether. The organic phase is dried over magnesium sulphate, filtered and then evaporated under reduced pressure. The residue obtained is recrystallised from toluene to yield the title product in the form of a pale yellow solid.

Melting point: 155-157° C.

Elemental microanalysis:

| | % C | % H | % N |
|---|---|---|---|
| calculated | 57.53 | 4.83 | 3.05 |
| found | 57.53 | 4.83 | 3.06 |

### EXAMPLE 6

### N-(2-{7-Methoxy-3-[3-methoxymethyl)phenyl]-1-naphthyl}ethyl)-acetamide

The compound obtained in Example 4 (0.1 g; 0.24 mmol), dissolved beforehand in 2 ml of methanol, is added dropwise to 10 ml of a freshly prepared solution of sodium methanolate (0.012 g; 0.48 mmol). The mixture is heated at the boil for 4 hours. After cooling, the methanol is evaporated off under reduced pressure and the residue is taken up in water and extracted with ether. The organic phase is dried over magnesium sulphate, filtered, and then evaporated under reduced pressure. The residue obtained is recrystallised from 95° ethanol to yield the title product in the form of a white solid.

Melting point: 86-87° C.

Elemental microanalysis:

| | % C | % H | % N |
|---|---|---|---|
| calculated | 76.01 | 6.93 | 3.85 |
| found | 75.37 | 6.92 | 3.82 |

### EXAMPLE 7

### N-(2-{3-[3-(Aminomethyl)phenyl]-7-methoxy-1-naphthyl}ethyl)-acetamide hydrochloride

### Step A: N-{2-[3-(3-Cyanophenyl)-7-methoxy-1-naphthyl]ethyl}acetamide

The procedure is as in Step A of Example 1, with the replacement of 2-formylphenylboronic acid with 2-cyanophenylboronic acid. The title compound is purified by chromatography on silica gel (acetone/hexane: 4/6) and obtained in the form of a white solid after recrystallisation from 95° ethanol.

Melting point: 141-143° C.

### Step B: N-(2-{3-[3-(Aminomethyl)phenyl]-7-methoxy-1-naphthyl}ethyl)-acetamide hydrochloride

The compound obtained in Step A (1.2 g; 3.5 mmol) is dissolved in 100 ml of methanol.

The solution is poured into an autoclave, and then 0.5 g of Raney nickel is added and the solution is saturated with ammonia gas. Hydrogen is introduced until a pressure of 50 bars is reached, and the reaction mixture is stirred for 12 hours at 60° C. The autoclave is cooled to ambient temperature, the Raney nickel is filtered off and the methanol is evaporated off under reduced pressure. The residue is taken up in ethyl ether and a solution of ethyl ether saturated with HCl gas is added dropwise until a precipitate is obtained. The precipitate is then suction-filtered off and recrystallised from isopropanol.

Melting point: 239-241° C.

### EXAMPLE 8

### N-(2-{3-[3-(Hydroxymethyl)phenyl]-7-methoxy-1-naphthyl}ethyl)-propanamide

### Step A: Methyl 3-{6-methoxy-4-[2-(propionylamino)ethyl]-2-naphthyl}benzoate

The procedure is as in Step A of Example 3, starting from the compound obtained in Preparation 2 and 3-(methoxycarbonyl)phenylboronic acid. The title compound is obtained in the form of a white solid after recrystallisation from 95° ethanol.

Melting point: 113-115° C.

Elemental microanalysis:

| | % C | % H | % N |
|---|---|---|---|
| calculated | 73.64 | 6.44 | 3.58 |
| found | 73.70 | 6.44 | 3.58 |

### Step B: N-(2-{3-[3-(Hydroxymethyl)phenyl]-7-methoxy-1-naphthyl}ethyl)-propanamide

The procedure is as in Step B of Example 3, starting from the compound obtained in Step A. The title compound is obtained in the form of a white solid after recrystallisation from 95° ethanol.

Melting point: 135-137° C.

### EXAMPLE 9

### N-(2-{3-[3-(Hydroxymethyl)phenyl]-7-methoxy-1-naphthyl}ethyl)-butanamide

### Step A: Methyl 3-{4-[2-(butyrylamino)ethyl]-6-methoxy-2-naphthyl}benzoate

The procedure is as in Step A of Example 1, starting from the compound obtained in Preparation 3 and with the replacement of 2-formylphenylboronic acid with (3-methoxycarbonyl)phenylboronic acid. The title compound is obtained in the form of a white solid after recrystallisation from 95° ethanol.

Melting point: 86-88° C.

Elemental microanalysis:

| | % C | % H | % N |
|---|---|---|---|
| calculated | 74.05 | 6.71 | 3.45 |
| found | 73.93 | 6.77 | 3.64 |

### Step B: N-(2-{3-[3-(Hydroxymethyl)phenyl]-7-methoxy-1-naphthyl}ethyl)-butanamide

The procedure is as in Step B of Example 3, starting from the compound obtained in

Step A. The title compound is obtained in the form of a white solid after recrystallisation from 95° ethanol.

Melting point: 113-115° C.

Elemental microanalysis:

| | % C | % H | % N |
|---|---|---|---|
| calculated | 76.36 | 7.21 | 3.71 |
| found | 76.21 | 7.15 | 3.72 |

### EXAMPLE 10

### N-(2-{3-[3-(Hydroxymethyl)phenyl]-7-methoxy-1-naphthyl}ethyl)-cyclobutanecarboxamide

### Step A: Methyl 3-(4-{2-[(cyclobutylcarbonyl)amino]ethyl}-6-methoxy-2-naphthyl)-benzoate

The procedure is as in Step A of Example 3, starting from the compound obtained in Preparation 4. The title compound is obtained in the form of a white solid after purification by chromatography on silica gel (acetone/cyclohexane: 3/7) followed by recrystallisation from 95° ethanol.

Melting point: 128-130° C.

Elemental microanalysis

| | % C | % H | % N |
|---|---|---|---|
| calculated | 74.80 | 6.52 | 3.35 |
| found | 74.55 | 6.48 | 3.32 |

### Step B: N-(2-{3-[3-(Hydroxymethyl)phenyl]-7-methoxy-1-naphthyl}ethyl)-cyclobutanecarboxamide

The procedure is as in Step B of Example 3, starting from the compound obtained in Step A. The title compound is obtained in the form of a white solid after recrystallisation from 95° ethanol.

Melting point: 131-133° C.

Elemental microanalysis:

| | % C | % H | % N |
|---|---|---|---|
| calculated | 77.09 | 6.99 | 3.60 |
| found | 76.98 | 7.05 | 3.53 |

### EXAMPLE 11

### 1-(2-{3-[3-(Hydroxymethyl)phenyl]-7-methoxy-1-naphthyl}ethyl)-2 pyrrolidinone

### Step A: Methyl 3-{6-methoxy-4-[2-(2-oxo-1-pyrrolidinyl)ethyl]-2-naphthyl}benzoate

The procedure is as in Step A of Example 3, starting from the compound obtained in Preparation 5. The title compound is obtained in the form of a white solid after purification by chromatography on silica gel (acetone/cyclohexane: 3/7) followed by recrystallisation from 95° ethanol.

Melting point: 110-112° C.

Elemental microanalysis:

| | % C | % H | % N |
|---|---|---|---|
| calculated | 74.42 | 6.25 | 3.47 |
| found | 74.09 | 6.29 | 3.63 |

### Step B: 1-(2-{3-[3-(Hydroxymethyl)phenyl]-7-methoxy-1-naphthyl}ethyl)-2-pyrrolidinone

The procedure is as in Step B of Example 3, starting from the compound obtained in

Step A. The title compound is obtained in the form of a white solid after recrystallisation from 95° ethanol.

Melting point: 129-131° C.

### Pharmacological Study

### EXAMPLE A

### Acute Toxicity Study

Acute toxicity was evaluated after oral administration to groups each comprising 8 mice (26±2 grams). The animals were observed at regular intervals during the course of the first day, and daily for the two weeks following treatment. The LD₅₀ (the dose that causes the death of 50% of the animals) was evaluated and demonstrated the low toxicity of the compounds of the invention.

### EXAMPLE B

### Melatonin Receptor Binding Study on Pars tuberalis Cells of Sheep

Melatonin receptor binding studies of the compounds of the invention were carried out according to conventional techniques on Pars tuberalis cells of sheep. The Pars tuberalis of the adenohypophysis is in fact characterised in mammals by a high density of melatonin receptors (Journal of Neuroendocrinology, 1, pp. 1-4, 1989).

Protocol

1) Sheep Pars tuberalis membranes are prepared and used as target tissue in saturation experiments to determine the binding capacities and affinities for 2-[¹²⁵I]-iodomelatonin.

2) Sheep Pars tuberalis membranes are used as target tissue in competitive binding experiments using the various test compounds in comparison with melatonin.

Each experiment is carried out in triplicate and a range of different concentrations is tested for each compound. The results, after statistical processing, enable the binding affinities of the compound tested to be determined.

Results

The compounds of the invention appear to have a strong affinity for melatonin receptors.

### EXAMPLE C

1. Study of Binding to Melatonin Receptors MT₁ and MT₂

The MT₁ or MT₂ receptor binding experiments are carried out using 2-[¹²⁵I]-iodomelatonin as reference radioligand. The radioactivity retained is determined using a liquid scintillation counter.

Competitive binding experiments are then carried out in triplicate using the various test compounds. A range of different concentrations is tested for each compound. The results enable the binding affinities of the compounds tested (Kᵢ) to be determined.

2. Study of Binding to Melatonin site MT₃

The experiments of binding to MT₃ sites are carried out on hamster brain membranes using 2-[¹²⁵I]-iodomelatonin as radioligand. The membranes are incubated for 30 minutes with 2-[¹²⁵I]-iodomelatonin at a temperature of 4° C. and at different concentrations of the compounds being tested. After incubation, the membranes are rapidly filtered and then washed with cold buffer using a filtration system. The radioactivity retained is measured by a scintillation counter. The IC₅₀ values (concentration that inhibits the specific binding by 50%) are calculated from competition curves according to a non-linear regression model.

Thus, the Kᵢ values found for the compounds of the invention show binding for one or another of the melatoninergic binding sites, those values being ≦10 μM.

By way of example, the compound of Example 2 has a Kᵢ of 0.36 nM in relation to the MT₂ site and the compound of Example 7 has a Kᵢ of 3.40 nM in relation to the MT₂ site.

In addition, the compound obtained in Step A of Example 2 has a Kᵢ of 0.42 nM in relation to the MT₂ site.

### EXAMPLE D

### Action of the Compounds of the Invention on the Circadian Rhythms of Locomotive Activity of the Rat

The involvement of melatonin in influencing the majority of physiological, biochemical and behavioural circadian rhythms by day/night alternation has made it possible to establish a pharmacological model for research into melatoninergic ligands.

The effects of the compounds are tested in relation to numerous parameters and, in particular, in relation to the circadian rhythms of locomotive activity, which are a reliable indicator of the activity of the endogenous circadian clock.

In this study, the effects of such compounds on a particular experimental model, namely the rat placed in temporal isolation (permanent darkness), are evaluated.

Experimental Protocol

One-month-old male rats are subjected, as soon as they arrive at the laboratory, to a light cycle of 12 hours of light per 24 hours (LD 12:12).

After 2 to 3 weeks' adaptation, they are placed in cages fitted with a wheel connected to a recording system in order to detect the phases of locomotive activity and thus monitor the nychthemeral (LD) or circadian (DD) rhythms.

As soon as the rhythms recorded show a stable pattern in the light cycle LD 12:12, the rats are placed in permanent darkness (DD).

Two to three weeks later, when the free course (rhythm reflecting that of the endogenous clock) is clearly established, the rats are given a daily administration of the compound to be tested.

The observations are made by means of visualisation of the rhythms of activity:
influence of the light rhythm on the rhythms of activity,
disappearance of the influence on the rhythms in permanent darkness,
influence by the daily administration of the compound; transitory or durable effect.

A software package makes it possible:
to measure the duration and intensity of the activity, the period of the rhythm of the animals during free course and during treatment,
to demonstrate by spectral analysis the existence of circadian and non-circadian (for example ultradian) components, where present.
   Results

The compounds of the invention clearly appear to enable powerful action on the circadian rhythm via the melatoninergic system.

### EXAMPLE E

### Light/Dark Cages Test

The compounds of the invention are tested on a behavioural model, the light/dark cages test, which enables the anxiolytic activity of the compounds to be revealed.

The equipment comprises two polyvinyl boxes covered with Plexiglas. One of the boxes is in darkness. A lamp is placed above the other box, yielding a light intensity of approximately 4000 lux at the centre of the box. An opaque plastic tunnel separates the light box from the dark box. The animals are tested individually for a session of 5 minutes. The floor of each box is cleaned between each session. At the start of each test, the mouse is placed in the tunnel, facing the dark box. The time spent by the mouse in the illuminated box and the number of passages through the tunnel are recorded after the first entry into the dark box.

After administration of the compounds 30 minutes before the start of the test, the compounds of the invention significantly increase the time spent in the illuminated cage and the number of passages through the tunnel, which demonstrates the anxiolytic activity of the compounds of the invention.

### EXAMPLE F

### Activity of the Compounds of the Invention on the Caudal Artery of the Rat

The compounds of the invention were tested in vitro on the caudal artery of the rat. Melatoninergic receptors are present in those vessels, thus providing a relevant pharmacological model for studying melatoninergic ligand activity. The stimulation of the receptors can induce either vasoconstriction or vasodilation depending upon the arterial segment studied.

Protocol

One-month-old rats are accustomed to a light/dark cycle of 12 h/12 h during a period of 2 to 3 weeks.

After sacrifice, the caudal artery is isolated and maintained in a highly oxygenated medium. The arteries are then cannulated at both ends, suspended vertically in an organ is chamber in a suitable medium and perfused via their proximal end. The pressure changes in the perfusion flow enable evaluation of the vasoconstrictive or vasodilatory effect of the compounds.

The activity of the compounds is evaluated on segments that have been pre-contracted by phenylephrine (1 μM). A concentration/response curve is determined non-cumulatively by the addition of a concentration of the test compound to the pre-contracted segment. When the effect observed reaches equilibrium, the medium is changed and the preparation is left for 20 minutes before the addition of the same concentration of phenylephrine and a further concentration of the test compound.

Results

The compounds of the invention significantly modify the diameter of caudal arteries pre-constricted by phenylephrine.

### EXAMPLE G

### Pharmaceutical Composition: Tablets

| | |
|---|---|
| 1000 tablets each containing a dose of 5 mg of N-[(2-{3-[3- | 5 g |
| (hydroxymethyl)phenyl]-7-methoxy-1-napthyl}ethyl)acetamide | |
| (Example 2) | |
| wheat starch | 20 g |
| maize starch | 20 g |
| lactose | 30 g |
| magnesium stearate | 2 g |
| silica | 1 g |
| hydroxypropyl cellulose | 2 g |

1. Compounds of formula (I): 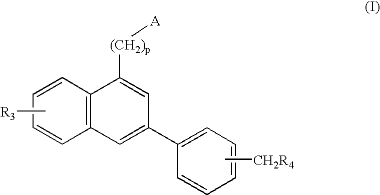
wherein:
A represents a grouping 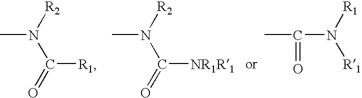
(wherein R₁ and R′₁, which may be identical or different, each represents a linear or branched (C₁-C₆)alkyl group, a linear or branched (C₂-C₆)alkenyl group, a linear or branched (C₂-C₆)alkynyl group, a (C₃-C₈)cycloalkyl group, a (C₃-C₈)cycloalkyl-(C₁-C₆)alkyl group in which the alkyl moiety may be linear or branched, an aryl group, an aryl-(C₁-C₆)alkyl group in which the alkyl moiety may be linear or branched, a heteroaryl group or a heteroaryl-(C₁-C₆)alkyl group in which the alkyl moiety may be linear or branched,
and R₂ represents a hydrogen atom or a linear or branched (C₁-C₆)alkyl group,
it being possible, additionally, for R₁ and R₂ together to form a linear or branched alkylene chain containing from 3 to 6 carbon atoms),
R₃ represents a linear or branched (C₁-C₆)alkoxy group,
R₄ represents a halogen atom, a hydroxy group, a linear or branched (C₁-C₆)alkoxy group or an amino group optionally substituted by one or two linear or branched (C₁-C₆)alkyl groups,
p is 1, 2 or 3,
it being understood that:
“aryl” denotes a phenyl, naphthyl or biphenyl group,
“heteroaryl” denotes any mono- or bi-cyclic aromatic group containing from 1 to 3 hetero atoms selected from oxygen, sulphur and nitrogen,
wherein the aryl and heteroaryl groups so defined may be substituted by from 1 to 3 groups selected from linear or branched (C₁-C₆)alkyl, linear or branched (C₁-C₆)alkoxy, hydroxy, carboxy, formyl, nitro, cyano, linear or branched polyhalo(C₁-C₆)alkyl, alkoxycarbonyl and halogen atoms,
their enantiomers and diastereoisomers, and addition salts thereof with a pharmaceutically acceptable acid or base.

2. Compounds of formula (I) according to claim 1, wherein A represents a grouping 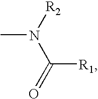
their enantiomers and diastereoisomers, and addition salts thereof with a pharmaceutically acceptable acid or base.

3. Compounds of formula (I) according to claim 1, wherein R₁ represents a linear or branched (C₁-C₆)alkyl group, their enantiomers and diastereoisomers, and addition salts thereof with a pharmaceutically acceptable acid or base.

4. Compounds of formula (I) according to claim 1, wherein R₁ represents a linear or branched (C₃-C₈)cycloalkyl group, their enantiomers and diastereoisomers, and addition salts thereof with a pharmaceutically acceptable acid or base.

5. Compounds of formula (I) according to claim 1, wherein R₂ represents a hydrogen atom, their enantiomers and diastereoisomers, and addition salts thereof with a pharmaceutically acceptable acid or base.

6. Compounds of formula (I) according to claim 1, wherein p is 2, their enantiomers and diastereoisomers, and addition salts thereof with a pharmaceutically acceptable acid or base.

7. Compounds of formula (I) according to claim 1, wherein R₃ represents a methoxy group, their enantiomers and diastereoisomers, and addition salts thereof with a pharmaceutically acceptable acid or base.

8. Compounds of formula (I) according to claim 1, wherein R₄ represents an OH group, their enantiomers and diastereoisomers, and addition salts thereof with a pharmaceutically acceptable acid or base.

9. Compounds of formula (I) according to claim 1, wherein R₄ represents an OMe group, their enantiomers and diastereoisomers, and addition salts thereof with a pharmaceutically acceptable acid or base.

10. Compounds of formula (I) according to claim 1, wherein R₄ represents an NH₂ group, their enantiomers and diastereoisomers, and addition salts thereof with a pharmaceutically acceptable acid or base.

11. Compounds of formula (I) according to claim 1, wherein R₄ represents a halogen atom, their enantiomers and diastereoisomers, and addition salts thereof with a pharmaceutically acceptable acid or base.

12. Compounds of formula (I) according to claim 1, wherein the —CH₂R₄ group is in the 3 position (meta) on the phenyl group, and addition salts thereof with a pharmaceutically acceptable acid or base.

13. Compound of formula (I) according to claim 1, which is N-(2-{3-[3-(hydroxymethyl)phenyl]-7-methoxy-1-naphthyl} ethyl)acetamide, and addition salts thereof with a pharmaceutically acceptable acid or base.

14. Compound of formula (I) according to claim 1, which is N-(2-{3-[3-(aminomethyl)phenyl]-7-methoxy-1-naphthyl}ethyl)acetamide, and addition salts thereof with a pharmaceutically acceptable acid or base.

15. Process for the preparation of compounds of formula (I) according to claim 1, characterised in that there is used as starting material a compound of formula (II): 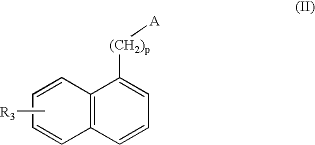
wherein A, p and R₃ are as defined for formula (I), which is subjected to the action of bromine to yield a compound of formula (III): 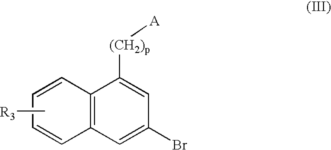
wherein A, p and R₃ are as defined hereinabove, which is condensed, in the presence of palladium acetate or tetrakis(triphenylphosphine)palladium, with a compound of formula (IV): 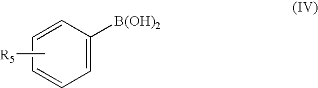
wherein R₅ represents a linear or branched (C₁-C₆)alkoxycarbonyl group, a formyl group or a cyano group, to yield a compound of formula (V): 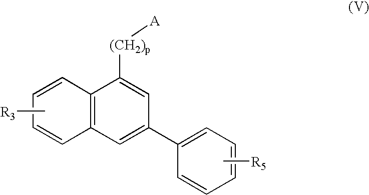
wherein A, p, R₃ and R₅ are as defined hereinabove,
which compound of formula (V),
when R₅ represents a CN group, is subjected to the action of Raney nickel to obtain a compound of formula (I/a), a particular case of the compounds of formula (I): 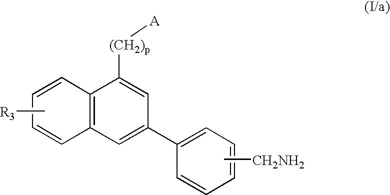
wherein A, p and R₃ are as defined hereinabove,
which compound of formula (I/a) may be subjected to the action of one or more alkylating agents to yield a compound of formula (I/b), a particular case of the compounds of formula (I): 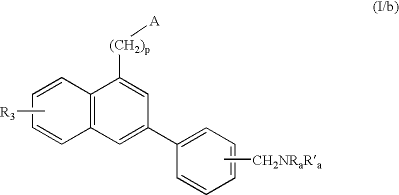
wherein A, p and R₃ are as defined hereinabove, Rₐ represents an alkyl group and R′ₐ represents a hydrogen atom or an alkyl group,
when R₅ represents a formyl group, is subjected to the action of NaBH₄ or triethylsilane and, when R₅ represents an alkoxycarbonyl group, is subjected to the action of LiAlH₄, to yield a compound of formula (I/c), a particular case of the compounds of formula (I): 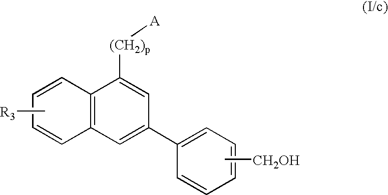
wherein A, p and R₃ are as defined hereinabove,
which compound of formula (I/c) is subjected to the action of a hydrohalic acid to yield a compound of formula (I/d), a particular case of the compounds of formula (I): 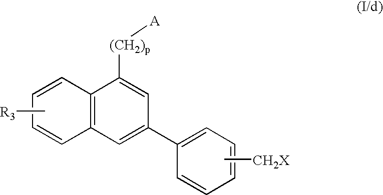
wherein A, p and R₃ are as defined hereinabove and X represents a halogen atom,
or which compound of formula (I/c) is subjected to the action of an alcoholate to yield a compound of formula (I/e), a particular case of the compounds of formula (I): 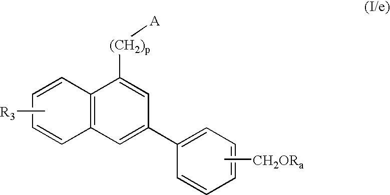
wherein A, p, R₃ and Rₐ are as defined hereinabove,
the compounds (I/a) to (I/e) constituting the totality of the compounds of formula (I), which compounds may be purified according to a conventional separation technique, are converted, if desired, into addition salts with a pharmaceutically acceptable acid or base, and are optionally separated into their isomers according to a conventional separation technique.

16. Compounds of formula (V′): 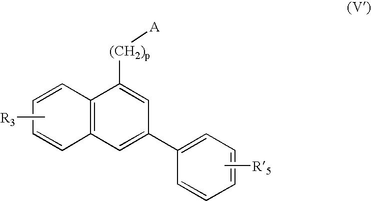
A represents a grouping 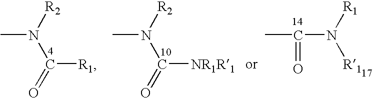
(wherein R₁ and R′₁, which may be identical or different, each represents a linear or branched (C₁-C₆)alkyl group, a linear or branched (C₂-C₆)alkenyl group, a linear or branched (C₂-C₆)alkynyl group, a (C₃-C₈)cycloalkyl group, a (C₃-C₈)cycloalkyl-(C₁-C₆)alkyl group in which the alkyl moiety may be linear or branched, an aryl group, an aryl-(C₁-C₆)alkyl group in which the alkyl moiety may be linear or branched, a heteroaryl group or a heteroaryl-(C₁-C₆)alkyl group in which the alkyl moiety may be linear or branched,
and R₂ represents a hydrogen atom or a linear or branched (C₁-C₆)alkyl group,
it being possible, additionally, for R₁ and R₂ together to form a linear or branched alkylene chain containing from 3 to 6 carbon atoms),
R₃ represents a linear or branched (C₁-C₆)alkoxy group,
R₁₅ represents a linear or branched (C₁-C₆)alkoxycarbonyl group or a formyl group, their enantiomers and diastereoisomers, and addition salts thereof with a pharmaceutically acceptable acid or base, for use as synthesis intermediates for the preparation of compounds of formula (I) but also as melatoninergic receptor ligands.

17. Pharmaceutical compositions comprising compounds of formula (I) according to any one of claims 1 to 14 or compounds of formula (V′) according to claim 16, or one of their addition salts with a pharmaceutically acceptable acid or base, in combination with one or more pharmaceutically acceptable excipients.

18. Pharmaceutical compositions according to claim 17 for use in the manufacture of medicaments for the treatment of disorders of the melatoninergic system.

19. Pharmaceutical compositions according to claim 17 for use in the manufacture of medicaments for the treatment of sleep disorders, stress, anxiety, seasonal affective disorders or severe depression, cardiovascular pathologies, pathologies of the digestive system, insomnia and fatigue due to jetlag, schizophrenia, panic attacks, melancholia, appetite disorders, obesity, insomnia, psychotic disorders, epilepsy, diabetes, Parkinson's disease, senile dementia, various disorders associated with normal or pathological ageing, migraine, memory losses, Alzheimer's disease, cerebral circulation disorders, and also sexual dysfunctions, and as inhibitors of ovulation, immunomodulators and in the treatment of cancers.
